(19) United States
(12) Patent Application Publication
(10) Pub. No.: US 2018/0272145 A1
(43) Pub. Date: Sep. 27, 2018

(54) WCD WITH SEPARABLE ECG ACQUISITION FEATURES

(71) Applicant: West Affum Holdings Corporation, Grand Cayman (KY)

(72) Inventors: Douglas K. Medema, Everett, WA (US); Joseph L. Sullivan, Kirkland, WA (US); David P. Finch, Bothell, WA (US)

(21) Appl. No.: 15/924,046

(22) Filed: Mar. 16, 2018

Related U.S. Application Data
(60) Provisional application No. 62/472,511, filed on Mar. 16, 2017.

(51) Int. Cl. A61N 1/39; A61B 5/0408; A61N 1/04

(52) U.S. Cl. CPC A61N 1/3904; A61N 1/3937; A61N 1/0484; A61B 5/04085; A61N 1/3987

(57) ABSTRACT

A superior wearable cardioverter defibrillator is disclosed. Embodiments provide one or more remote sensors (e.g., ECG electrodes) that are separable from the rest of the WCD system, which holds the defibrillation electrodes on the body and may also hold a preamplifier for the electrodes. This feature enables

ECG electrodes (or other physiological sensors) to be more securely affixed to the patient's body without adversely affecting the patient's desire to don the rest of the WCD.

### CROSS REFERENCE TO RELATED APPLICATIONS

This patent application claims the benefit of and priority to U.S. Provisional Patent Application Ser. No. 62/472,511 filed on Mar. 16, 2017, entitled “Wearable Cardiac Defibrillator With Separable ECG Acquisition Features,” the disclosure of which is hereby incorporated by reference for all purposes.

### TECHNICAL FIELD

The disclosed subject matter pertains generally to the area of medical devices, and more specifically to the area of wearable cardiac defibrillators.

### BACKGROUND INFORMATION

Some people, such as those who have had a myocardial infarction (MI), are at increased risk of sudden cardiac arrest (SCA). But for a number of reasons, they may not be considered good candidates for implantable cardiac defibrillators. For example, those who have had an MI are not good candidates for a period of time after recovering from the MI.

To address that concern, special devices have been developed to service a patient either until the patient can receive an implantable cardiac defibrillator (if that is intended) or if the patient cannot receive an implantable cardiac defibrillator. These special devices are referred to as wearable cardioverter defibrillators or sometimes wearable cardiac defibrillators (“WCDs”). A WCD is typically implemented as a unitary garment, such as a harness or vest, that the patient wears. The WCD includes electronic components, such as a defibrillator and electrodes, integrated within the garment.

When the patient wears the WCD, the electrodes make electrical contact with the patient's skin, and therefore can help detect the patient's heart rhythm. In this way, the WCD monitors the electrocardiogram (ECG) of the patient. It can sense if the patient appears to be experiencing an SCA. If a shockable heart arrhythmia is detected, the defibrillator delivers an electric shock to the patient, hopefully eliminating the arrhythmia and returning an adequate heart rhythm.

For reasons that should be apparent, accurate detection of an SCA is an important aspect of the effectiveness of the WCD. The downside of delivering an unnecessary defibrillation shock to a patient is exceeded only by the downside of not delivering a necessary defibrillation shock.

Improvements to wearable cardioverter defibrillators are constantly being sought by those skilled in the art.

### SUMMARY OF EMBODIMENTS

This disclosure is directed to a superior wearable cardioverter defibrillator. Embodiments provide one or more remote sensors (e.g., ECG electrodes) that are separable from the rest of the WCD system, which holds the defibrillation electrodes on the body and may also hold a preamplifier for the electrodes. This feature enables ECG electrodes (or other physiological sensors) to be more securely affixed to the patient's body without adversely affecting the patient's desire to don the rest of the WCD.

In certain embodiments, an additional separate component may be provided to keep the main electronics module in close proximity to the elements secured to the WCD garment. In certain embodiments, signals from ECG electrodes could be sent wirelessly or via a wire to either a preamplifier or the main electronics module of the WCD.

### BRIEF DESCRIPTION OF THE DRAWINGS

FIGS. 1A and 1B are conceptual diagrams of a wearable cardioverter defibrillator system made in accordance with this disclosure.

FIG. 2 is another conceptual diagram of a patient having remote sensors, which form a part of a wearable cardioverter defibrillator system, affixed to the patient's body.

FIG. 3 is still another conceptual diagram of a patient having remote sensors, which form a part of a wearable cardioverter defibrillator system, affixed to the patient's body.

FIG. 4 is yet another conceptual diagram of a patient having remote sensors, which form a part of a wearable cardioverter defibrillator system, affixed to the patient's body.

FIG. 5 is a functional block diagram generally illustrating components of a wearable cardioverter defibrillator system made in accordance with the teachings of this disclosure.

### DETAILED DESCRIPTION

Generally stated, this disclosure is directed at improvements in monitoring a patient's condition (e.g., electrocardiogram) for an extended period of time in the application of a wearable cardiac defibrillator (WCD). This disclosure begins with an overview to describe, generally, aspects of several of the embodiments as distinguished from existing WCD technology. Next is a description of one example of a medical device that may be used in specific embodiments. Finally, unforeseen benefits realized by embodiments of the disclosure are discussed.

Overview of the Disclosure

Much has been learned in the pursuit of a superior WCD system. For instance, an important aspect of successful use of a WCD is the patient's compliance in continuously wearing the WCD except, for instance, while bathing. Existing WCDs use a single garment on which are mounted ECG electrodes, defibrillation electrodes, and Analog Front End (AFE) electronics. While the garment should keep the defibrillation electrodes in contact with the body, the main purpose of the garment is to hold the ECG electrodes in place against the body with sufficient pressure while minimizing the motion of the electrode on the skin. A single garment designed to hold the defibrillation electrodes and the AFE along with the ECG electrodes must be larger and stiffer than that required to hold the ECG electrodes alone. Both the size and stiffness work against minimizing motion of the ECG electrodes as well as keeping consistent pressure on them.

In addition, the single garment requires the ECG electrodes to be removed from the body every time the patient removes the garment. This results in poorer ECG signal quality because the signal quality is usually compromised during the first 10-30 minutes after the ECG electrodes make contact with the body. Finally, it is highly unlikely that a single garment system could be designed that could be worn while bathing. The electronics would be difficult to be made water proof and the garment material itself would need to be dried after bathing. Furthermore, a single garment is likely to cover large areas of the torso thus preventing normal bathing. Thus the current art has several disadvantages which result in decreased comfort for the patient, more alarm conditions the patient must deal with, and reduced utility since it must be taken off during bathing.

The disclosed embodiments overcome these deficiencies by separating the ECG electrodes from the rest of the WCD. In short, a WCD garment is provided that holds a substantial portion of the components of the WCD system (e.g., the defibrillator and defibrillator electrodes), and a separable component is provided which houses sensors, such as ECG electrodes. The WCD garment and the separable component are in detachable communication such that ECG signals collected by the ECG electrodes are delivered to components on the WCD garment. The WCD garment and the separable component may be either in wired or in wireless communication.

The present disclosure can be implemented in many different embodiments. Several illustrative embodiments will now be described in conjunction with the associated drawings, which form a part of this disclosure.

Description of Wearable Cardioverter Defibrillator

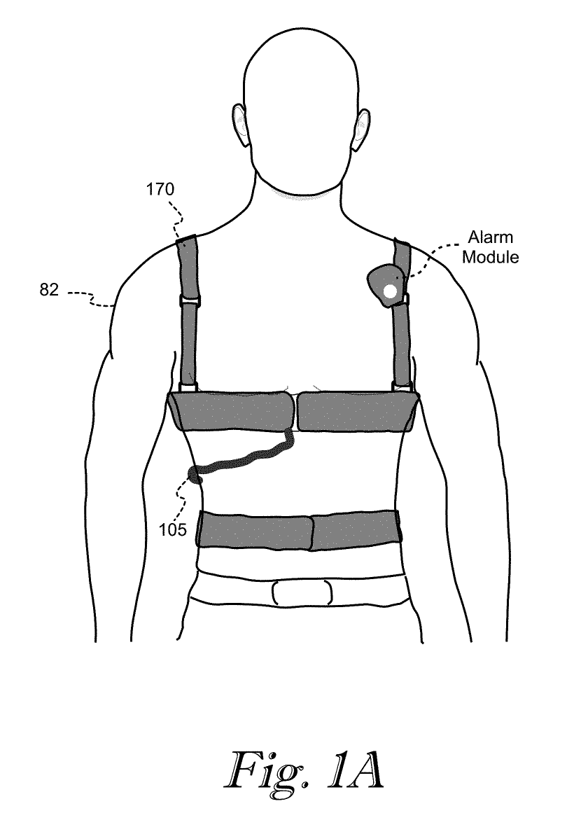

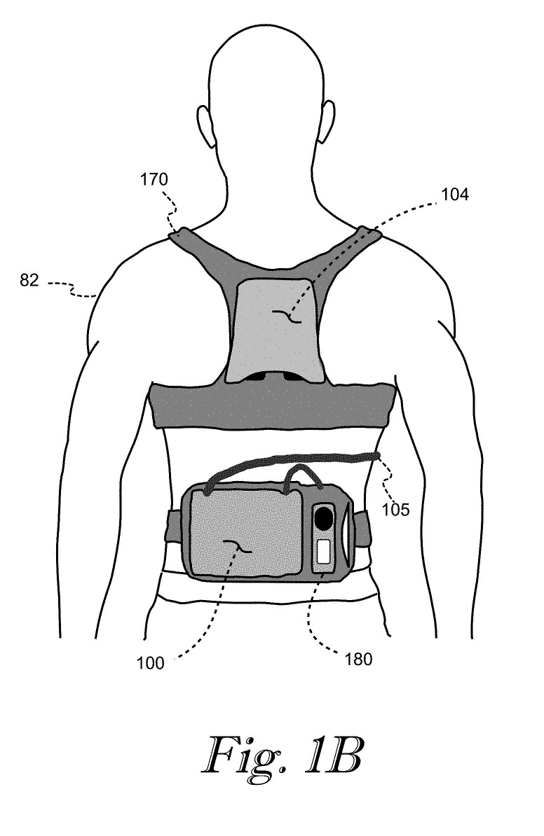

FIGS. 1A and 1B together provide a conceptual diagram generally showing components of a medical device that may be adapted to implement embodiments of this disclosure. In this particular example, the medical device is a wearable cardioverter defibrillator (WCD) system. FIG. 1A is a front view of the WCD system; and FIG. 1B is a rear view of the WCD system. FIGS. 1A and 1B may be collectively referred to as “FIG. 1.”

A patient 82, who is ambulatory, is shown in FIG. 1. Patient 82 may also be referred to as “wearer,” since the patient wears at least some components of the WCD system.

One component of the WCD system is a garment 170 that is wearable by patient 82. The garment 170 acts as a support structure for several of the other components of the WCD system. The garment 170 is a semi-rigid wearable element sufficient to support and contain at least some other components of the WCD system. The illustration shown in FIG. 1 is provided merely to describe concepts about the garment 170, and is not to be construed as limiting how garment 170 may be implemented in various embodiments, or how it is worn.

Garment 170 can be implemented in many different ways. For example, it may be implemented as a single component or as a combination of multiple components. In some embodiments, garment 170 includes a harness, one or more belts or straps, etc. In such embodiments, those items can be worn around the torso or hips, over the shoulder, or the like. In other embodiments, garment 170 includes a container or housing, which may be waterproof.

As shown in FIG. 1, the WCD system includes an external defibrillator 100, defibrillation electrodes 104, and electrode leads 105 which couple the defibrillator 100 to the defibrillation electrodes 104. In many embodiments, defibrillator 100 and defibrillation electrodes 104 are supported by garment 170. In other embodiments, defibrillator 100 may be supported by an ancillary article of clothing, such as the patient's belt.

The WCD system may also include a monitoring device (e.g., monitor 180) to monitor the patient 82, the patient's environment, or both. In many embodiments, the monitor 180 is coupled to one or more sensors, such as electrocardiogram electrodes. Using those sensors, the monitor 180 detects criteria upon which a shock/no-shock decision can be made.

In certain embodiments, monitor 180 is implemented as a component of the defibrillator 100. In other embodiments, monitor 180 may be implemented as a stand-alone monitoring device supported by the garment 170 or perhaps worn separately, such as on the patient's wrist or belt. In such cases, monitor 180 may be communicatively coupled with other components, which are coupled to garment 170. Such communication can be implemented by a communication module, as will be described below.

Optionally, the WCD system may include a fluid that can be deployed between the defibrillation electrodes and the patient's skin. The fluid can be conductive, such as by including an electrolyte, for making a better electrical contact between the electrode and the skin. Electrically speaking, when the fluid is deployed, the electrical impedance between the electrode and the skin is reduced. Mechanically speaking, the fluid may be in the form of a low-viscosity gel, so that it does not flow away, after it has been deployed. The fluid can be used for both defibrillation electrodes 509, and sensing electrodes 209.

The WCD system is configured to defibrillate the patient 82 by delivering an electrical shock (sometimes referred to as a pulse, defibrillation shock, therapy, or therapy shock) to the patient's heart through the patient's body. When defibrillation electrodes 104 make good electrical contact with the body of patient 82, defibrillator 100 can administer, via electrodes 104 a brief, strong electric shock through the patient's body. The shock is intended to go through and restart the patient's heart, in an effort to save the life of the patient 82.

In accordance with this disclosure, embodiments further provide remote sensors, such as ECG electrodes, which are in operative communication with the defibrillator 100. The remote sensors provide the defibrillator with physiological information upon which the shock/no-shock decision can be made. In further accordance with this disclosure, these remote sensors are either separate or separable from the garment 170.

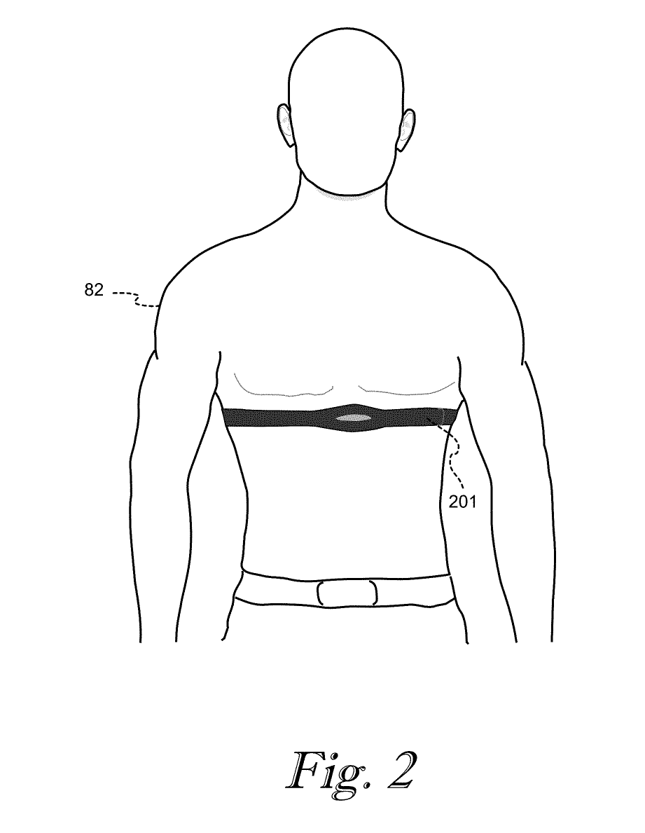

In most cases, the garment 170 is worn over the remote sensors (e.g., the mechanism that supports ECG electrodes). FIGS. 2-4 show illustrative embodiments of remote sensors without the outer garment 170 in place.

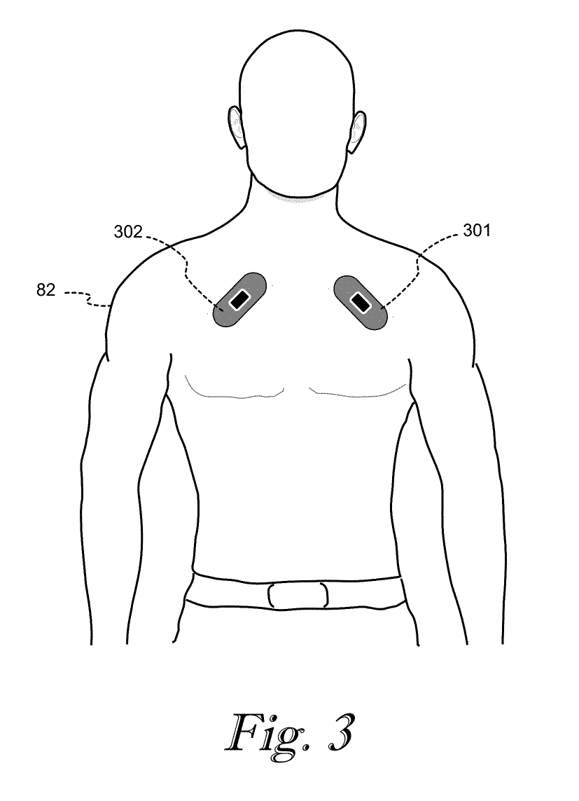

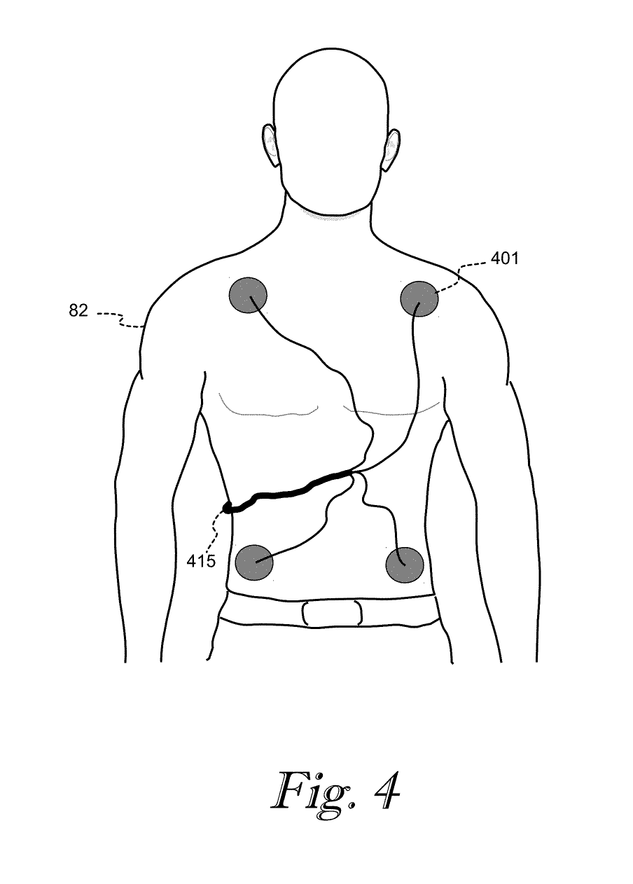

Turning now to FIG. 2, a supporting band 201 around the patient's midsection or torso may be used. In such an embodiment, ECG electrodes (or other physiological sensor) may held in place by the supporting band 201. Turning to FIG. 3, ECG electrodes 301, 302 may have an adhesive which is compatible with long term wear by the patient 82. ECG electrodes 301, 302 may be implemented with built-in wireless capability so that the ECG electrodes 301, 302 can communicate with defibrillator 100 wirelessly. FIG. 4 shows another embodiment using individual wired ECG electrodes which would be connected to the outer garment 170 through ECG leads.

The examples illustrated in FIG. 2-4 are conceptual and demonstrate the general concept that remote sensors may be implemented that are separable from the main outer garment 170 of the WCD system. This feature enables the WCD system to achieve certain unforeseen benefits, as will be discussed below. It should be appreciated that the particular number of remote sensors illustrated in FIGS. 2-4 is for purposes of disclosure only. Alternative embodiments are envisioned with any number of remote sensors without materially deviating from the teachings of this disclosure.

Electronic Components of the WCD

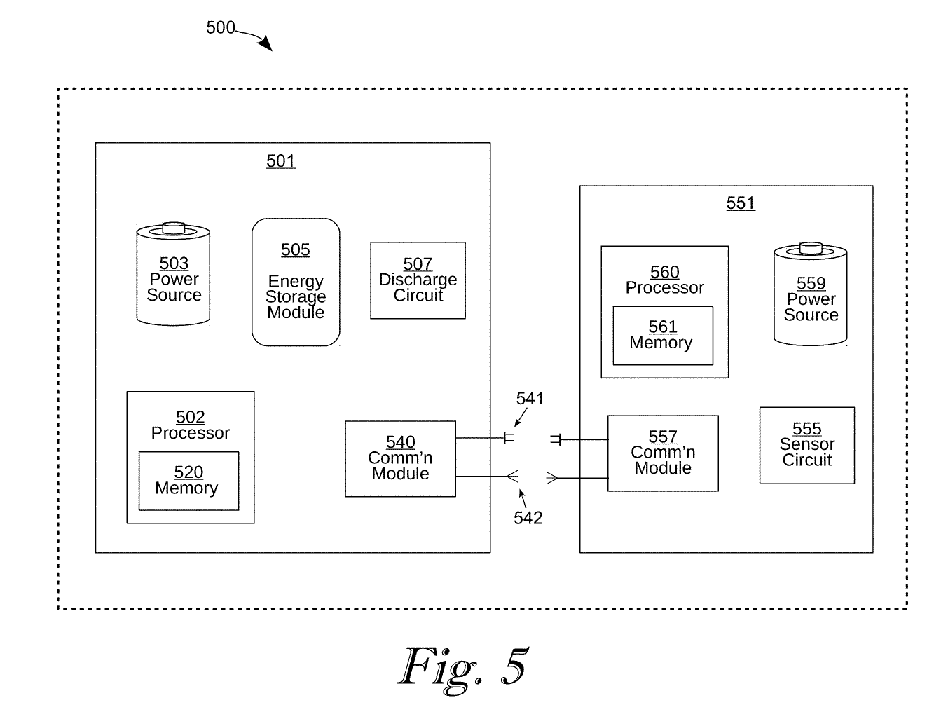

In the interest of complete disclosure, reference is now made to certain electrical components of a typical WCD system according to this disclosure. Turning to FIG. 5, functional components of one illustrative WCD system 500, in accordance with these teachings, are shown. Generally stated, the core operative components of the WCD system include a defibrillator 501 and a remote sensor 551. Each of those components will now be described, followed by a brief overview of the operation of the WCD system.

Defibrillator of the WCD System

The defibrillator 501 of the illustrative WCD includes at least a processor, a power source, an energy storage module, and a discharge circuit. The processor 502 may be implemented as a digital and/or analog processor, such as microprocessors and Digital Signal Processors (DSPs); microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The processor 502 may include, or have access to, a memory 520 that may be either volatile, nonvolatile, or some combination of the two. Computer executable instructions may be stored in the memory 520. The instructions generally provide functionality by defining methods as may be disclosed herein or understood by one skilled in the art in view of this disclosure. The memory may be implemented as Read-Only Memory (ROM), Random Access Memory (RAM), magnetic disk storage media, optical storage media, flash memory, any combination of these, or the like.

The power source 503 may be any type of electrical component sufficient to provide power, such as a battery. Other types of power source 503 could include an AC power override, for where AC power will be available, an energy storage capacitor, and so on. Appropriate components may also be included to provide for charging or replacing power source 503.

The defibrillator 501 may additionally include an energy storage module 505. Energy storage module 505 is where electrical energy is stored temporarily in the form of an electrical charge, when preparing it for discharge to administer a shock. Energy storage module 505 can be charged from power source 503 to the desired amount of energy. In typical implementations, module 505 includes a capacitor, which can be a single capacitor or a system of capacitors, and so on. In some embodiments, energy storage module 505 includes a device that exhibits high power density, such as an ultracapacitor.

The defibrillator component also includes a discharge circuit 507. When the processor 502 determines that a shock is appropriate (as described below), the processor 502 instructs the discharge circuit 507 to discharge the electrical charge stored in energy storage module 505 to the patient. When so controlled, the discharge circuit 507 causes the energy stored in energy storage module 505 to be discharged to defibrillation electrodes 509, so as to cause a shock to be delivered to the patient.

In accordance with this disclosure, defibrillator 501 also includes a communication module 540 configured to enable communication with remote components. In certain embodiments, communication module 540 includes a wireless communication facility to enable wireless communication between the defibrillator 501 and remote components. Examples of wireless communication that may be enabled include 802.11 (WiFi) communication, Bluetooth communication, Near Field Communication (NFC), infrared communication, or the like.

In other embodiments, communication module 540 includes a wired communication facility to enable wired communication. In such embodiments, defibrillator 501 may include a communication port 541 through which the wired communication may be effected. Examples of such wired communication may include a universal serial bus connector (e.g., USB-C, micro-USB, mini-USB, USB-A, or the like), a coaxial connector, an Ethernet connector, a 12-lead connector, or the like.

The communication module 540 enables the defibrillator 501 to receive input data from remote sensors. In this way, the processor 502 may receive sensory input data upon which it can base a shock/no-shock decision. In one specific example, communication module 540 enables wireless communication between a remote ECG sensor attached to the patient. In this way, the remote ECG sensor (e.g., remote sensor 551) can be more securely attached to the patient while remaining in operative communication with the defibrillator 501. Similarly, a detachable wired connection could be made between the remote ECG sensor and the communication module 540 to obtain a similar benefit.

Remote Sensor of the WCD System

The remote sensor 551 is another component of the WCD system 500. The remote sensor 551 includes at least a sensor circuit 555 and a communication module 557. The communication module 557 of the remote sensor 551 may function similar to the component of the same name described above (i.e., communication module 540). In other words, the communication module 557 of the remote sensor 551 may enable either wired or wireless communication between the remote sensor 551 and the defibrillator 501 (or any other component of the WCD system 500).

In wireless embodiments, the remote sensor 551 may further include a power source 559 and a remote processor 560. In such embodiments, the remote processor 560 is configured to control and manage the operation of the several components of the remote sensor 551 and to cause physiological signals to be transmitted from the remote sensor 551 to the defibrillator 501. In wired embodiments, both power and processing capability may be provided to the remote sensor 551 from the defibrillator 501 over the wired connection. Of course, the use of a wired connection does not foreclose the inclusion of a processor, or a power supply, or both on the remote sensor 551.

In many embodiments, remote sensor 551 is operative to sense one or more physiological conditions of the patient, such as the patient's heart rhythm. In such embodiments, the remote sensor 551 may be implemented, for example, as a wireless ECG electrode or sensor which can be securely affixed to the patient. In certain embodiments, Analog Front End (AFE) electronics may be included as part of the remote sensor 551 and transmit the data wirelessly to the main electronics module (e.g., defibrillator 501).

Sensor circuit 555 may be implemented as an ECG electrode as has been stated repeatedly. However, sensor circuit 555 may alternatively be implemented as one or more of various other sensors. Examples of such alternative sensors include mechanisms for monitoring the patient's blood oxygen level, blood flow, blood pressure, blood perfusion, pulsatile change in light transmission or reflection properties of perfused tissue, heart sounds, heart wall motion, breathing sounds and pulse. Examples of such sensors or transducers include a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on.

The sensor circuit 555 may alternatively or additionally include a position detector 560. Such a position detector can be configured to detect a location or movement of the patient. Such a position detector can be implemented in many ways as is known in the art, such as, for example, an accelerometer, a GPS location sensor, or the like. Environmental parameters may also be monitored, such as ambient temperature and pressure. Moreover, a humidity sensor may provide information as to whether it is likely raining. Still other sensor circuits 555 may be implemented which detect many other environmental criteria, as will be apparent to those skilled in the art.

Embodiments of the remote sensor 551 may be implemented in many ways. For instance, remote sensor 551 may be implemented as a monitor that adheres directly to a patient's body, such as is shown in FIGS. 2-4. Alternatively, remote sensor 551 may be implemented as a wearable device, such as a smartwatch or other wrist-worn band. In yet other alternatives, remote sensor 551 may be implemented as a component of a mobile device (e.g., a specially adapted cellular phone) which the patient may carry in the hand or, perhaps, in a pocket.

Operation of the WCD System

In operation, the patient attaches the remote sensor 551 as appropriate, e.g., adhesively to the patient's upper torso. The patient then dons the rest of the WCD system, such as garment 170 (FIG. 1). As the patient wears the WCD system, the remote sensor 551 of the preferred embodiment constantly provides the patient's ECG information to the defibrillator 501. In the event that the processor 502 detects that patient's ECG suggests that a life threatening arrhythmia is occurring, the processor 502 may trigger an alarm, providing the patient with an opportunity to override the shock should the patient be conscious and understand that the alarm represents a false positive. In the absence of a terminating signal from the patient, the processor 502 may instruct discharge circuit 507 to discharge the electrical charge that is stored in energy storage module 505 to the patient, hopefully thereby remedying the arrhythmia.

Benefits Realized by this Disclosure

Embodiments of the disclosure enable numerous benefits over existing technologies. Most of these benefits were unforeseen and have resulted in improvements to WCD systems which were not anticipated. Some of the benefits of this disclosure are enumerated below.

Implementations of this disclosure allow relative motion between the larger, heavier outer garment and the lighter-weight ECG electrode mechanism.

Embodiments also allow for placement of the ECG electrodes where the outer garment is unlikely to change the pressure applied to the electrodes. Both of these features result in reduced ECG artifacts due to ECG electrode movement.

With separable remote sensors (e.g., ECG electrodes), more robust attachment mechanisms (e.g., adhesives) can be employed to provide better ECG readings. The improvement in ECG detection and monitoring realized by this feature results in fewer false positives (e.g., alarms being sounded when no life threatening arrhythmia is actually occurring), which results in the patient having greater confidence that the WCD system will in fact operate properly if and when necessary.

The outer garment can be removed without affecting the integrity of the ECG electrode to skin interface. This feature decreases the time required between when the outer garment is put on and good signals are obtained, thereby reducing the amount of time, even slightly, that the patient is at high risk.

Embodiments of the disclosure enable sensors to be used which cover a smaller area of the body. This increases patient comfort and can feel less constricting.

Existing WCD systems require an extensive process for removing the electrodes and electronics before laundering and then re-assembling the system after laundering has been completed. Embodiments of the disclosure allow for the outer garment to be prepared for laundering with a much simpler process. In embodiments that use a wired connection between the WCD garment and the remote sensor, a single connection point allows for ECG electrodes to be more quickly disconnected and reconnected, thus simplifying the laundering process.

Waterproof remote sensors can be used which are more-securely affixed to the patient. of both instantiations thus allowing the patient to bathe with the electrodes in place. The amount of torso covered by the ECG electrodes is minimized allowing the patient more normal bathing practices.

Other embodiments may include combinations and sub-combinations of features described above or shown in the Figures, including, for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

In the foregoing description, numerous details have been set forth in order to provide a sufficient understanding of the described embodiments. In other instances, well-known features have been omitted or simplified to not unnecessarily obscure the description.

A person skilled in the art in view of this description will be able to practice the disclosed invention. The specific embodiments disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems. The following claims define certain combinations and subcombinations of elements, features, steps, and/or functions, which are regarded as novel and non-obvious. Additional claims for other combinations and subcombinations may be presented in this or a related document.

What is claimed is:

1. A medical device, comprising:
a garment on which is semi-permanently affixed an external defibrillator and defibrillator electrodes, the external defibrillator being connected to the defibrillator electrodes through defibrillator leads, the garment having sufficient structural integrity to support the external defibrillator and to maintain the defibrillator electrodes in an operative position, at least one component affixed to the garment having a communication module; and
a remote sensor having a sensor circuit operative to monitor a human heart rhythm, the remote sensor having a remote communication facility operative to communicate with the communication module of the garment over a communication link,
wherein the remote sensor is configured to transmit data pertaining to the human heart rhythm over the communication link when the communication link is active between the remote communication facility and the communication module, and
wherein the external defibrillator is configured to deliver an electric shock in the event that the data pertaining to the human heart rhythm suggests an arrhythmia.

2. The medical device recited in claim 1, wherein the external defibrillator further comprises a processor configured to analyze the data pertaining to the human heart rhythm and to make a determination whether the data pertaining to the human heart rhythm suggests the arrhythmia.

3. The medical device recited in claim 1, wherein the remote sensor further comprises a position detection component operative to identify a physical location of the remote sensor.

4. The medical device recited in claim 3, wherein the position detection component comprises an accelerometer.

5. The medical device recited in claim 3, wherein the position detection component comprises a GPS sensor.

6. The medical device recited in claim 1, further comprising a second remote sensor having a position detection component operative to identify a physical location of the remote sensor.

7. The medical device recited in claim 1, wherein the communication link comprises a wireless communication link.

8. The medical device recited in claim 1, wherein the communication link comprises a wired communication link.

9. The medical device recited in claim 1, wherein the communication facility comprises a wireless communication facility and wherein the remote sensor further comprises analog front end electronics to drive the sensor circuit.

10. The medical device recited in claim 1, wherein the sensor circuit comprises an ECG sensor.
